(19) United States
(12) Patent Application Publication
(10) Pub. No.: US 2024/0081676 A1
(43) Pub. Date: Mar. 14, 2024

(54) MEASURING DEVICE FOR ANALYZING A RESPIRATORY GAS FLOW

(71) Applicant: Loewenstein Medical Technology S.A., Luxembourg (LU)

(72) Inventors: Fritz MEHR, Merenberg (DE); Otto STROEBEL, Simmern (DE)

(21) Appl. No.: 18/272,161

(22) PCT Filed: Jan. 20, 2022
(86) PCT No.: PCT/EP2022/025019

§ 371 Date: Jul. 13, 2023

(30) Foreign Application Priority Data
Jan. 22, 2021 (DE) 10 2021 000 320.9

(51) Int. Cl. A61B 5/083; A61B 5/097; A61M 16/00; A61M 16/06

(52) U.S. Cl. CPC A61B 5/0836; A61B 5/097; A61M 16/0003; A61M 16/0605

(57) ABSTRACT

Measuring device (1) for analyzing a respiratory gas flow, comprising at least one measuring unit (4) and a cuvette (5), the cuvette (5) being releasably connected to the measuring unit (4) and being adapted and configured so that a respiratory gas flows through said cuvette, the measuring unit (4) having at least two sensor units (41, 42), at least one sensor unit (41) being configured to determine a respiratory gas flow and at least one sensor unit (42) being configured to determine a CO2 concentration in a respiratory gas, and the cuvette (5) comprising at least two sensor connections (51, 52) for connecting the sensor units (41, 42) for determining at least one respiratory gas flow and at least one CO2 concentration of a respiratory gas.

The invention relates to a measuring device for analyzing a respiratory gas flow, which can be analyzed by a measuring device simultaneously in respect of at least two parameters.

Particularly in the ventilation of preterms, a low dead space volume of the measuring apparatuses used for monitoring the respiration is important. Measuring apparatuses from the prior art for analyzing a respiratory gas flow in this case present approaches for reducing the size of the measuring cuvette per se and therefore also the dead space volume. However, the measuring apparatuses in this case generally comprise only individual sensor units for monitoring the respiratory gas flow of the living being, or patient. It is therefore necessary to connect a plurality of sensor units successively to corresponding connectors and cuvettes, or connections, as a result of which the dead space volume is increased considerably. Particularly in the ventilation of preterms in the clinical field, an excessively large dead space volume may result in the patient not being able to be supplied with sufficient fresh respiratory air and a significant amount of CO2 rebreathing being observable. Sometimes, certain sensors are therefore omitted, which may in turn have the effect that, for example, life-threatening changes are not registered and may be overlooked.

It is therefore an object of the present invention to provide a measuring device for effective and reliable respiratory gas flow analysis of a living being. The object is achieved by the measuring device according to the invention as claimed in claim 1.

The object is achieved by a measuring device for analyzing a respiratory gas flow, comprising at least one measuring unit and a cuvette, the cuvette being releasably connected to the measuring unit and being adapted and configured for a respiratory gas to flow through it, wherein the measuring unit comprises at least two sensor units, at least one sensor unit being configured to determine a respiratory gas flow rate and at least one sensor unit being configured to determine a CO2 concentration in a respiratory gas, and the cuvette comprises at least two sensor connectors for attaching the sensor units for determining at least one respiratory gas flow rate and at least one CO2 concentration of a respiratory gas.

In some embodiments, the measuring device is characterized in that the cuvette comprises at least three sensor connectors, the at least third sensor connector being a sensor connector for a sensor unit for determining a respiratory gas pressure. In this embodiment, the cuvette thus comprises a connector for a sensor unit for determining (or measuring) the CO2 concentration of the respiratory gas, the respiratory gas flow rate and the respiratory gas pressure.

In some embodiments, the measuring device is characterized in that the measuring unit comprises at least three sensor units, the at least third sensor unit being a sensor unit for determining the respiratory gas pressure. In this embodiment, the measuring unit thus respectively comprises a sensor unit for determining (or measuring) the CO2 concentration of the respiratory gas, the respiratory gas flow rate and the respiratory gas pressure.

In some embodiments, the measuring device is characterized in that the sensor unit for determining the CO2 concentration in the respiratory gas comprises at least one beam source, at least one detector unit, at least one mirror, at least two lenses and at least one prism.

In some embodiments, the measuring device is characterized in that at least two of the lenses are configured as Fresnel lenses and at least one of the two lenses is arranged on the at least one prism.

In some embodiments, the measuring device is characterized in that the sensor unit comprises at least two prisms, at least one of the at least two lenses being arranged on each prism.

In some embodiments, the measuring device is characterized in that the mirror is a concave mirror.

In some embodiments, the measuring device is characterized in that the mirror is coated for high reflection of beams in a wavelength range of from 3500 nm to 4600 nm, particularly in the wavelength range of 3910+/−100 nm and 4260+/−100 nm, preferably with gold.

In some embodiments, the measuring device is characterized in that the mirror is configured to be aspherical.

In some embodiments, the measuring device is characterized in that the mirror is configured to be anamorphic.

In some embodiments, the measuring device is characterized in that the detector unit comprises at least two detector faces.

In some embodiments, the measuring device is characterized in that at least one of the at least two detector faces is configured for detecting one of wavelengths in a range of from 3950 nm to 4550 nm, preferably in the wavelength range of 4260+/−100 nm, and at least one of the at least two detector faces is configured for detecting wavelengths in a range of from 3600 nm to 4200 nm, preferably in the wavelength range of 3910+/−100 nm.

In some embodiments, the measuring device is characterized in that at least one of the at least two detector faces is configured as a measuring detector and at least one of the at least two detector faces is configured as a comparison detector, the at least two detector faces having the same size, and the detector face as a measuring detector being configured for detecting wavelengths in a range of from 3950 nm to 4550 nm, preferably in the wavelength range of 4260+/−100 nm.

In some embodiments, the measuring device is characterized in that the beam source is an infrared beam source, preferably an incandescent lamp. In this case, both a conventional incandescent lamp and an incandescent lamp specially configured as an infrared beam source may be envisioned.

In some embodiments, the measuring device is characterized in that at least one prism is configured as an inverting prism, preferably as a roof prism.

In some embodiments, the measuring device is characterized in that at least one prism is materially connected to at least one of the at least two lenses.

In some embodiments, the measuring device is characterized in that at least one prism is produced in one piece with at least one of the at least two lenses. In some embodiments, the measuring device comprises at least two prisms, each of the two prisms respectively being produced in one piece with one of the at least two lenses.

In some embodiments, the measuring device is characterized in that the cuvette is arranged between one of the at least two lenses and the at least one prism.

In some embodiments, the measuring device is characterized in that the cuvette is arranged between two prisms, the two prisms being arranged between two lenses, so that a sequence of lens, prism, cuvette, prism, lens is obtained.

In some embodiments, the measuring device is characterized in that the mirror, the beam source, the lenses, the prisms of the sensor unit and the sensor connector of the cuvette for attaching the sensor unit for determining the CO2 concentration are not coated with an antireflection coating.

In some embodiments, the measuring device is characterized in that the overall transmission of the optics consisting of the mirror, the beam source, the lenses and the prisms of the sensor unit as well as of the cuvette is more than 50%, preferably more than 60%.

In some embodiments, the measuring device is characterized in that the prisms, the lenses and the cuvette are produced from a plastic, the plastic having, with a material thickness of 2 mm, a transmission of infrared beams, preferably in the wavelength range of between 3910+/−100 nm and 4260+/−100 nm, of more than 85%, preferably more than 90%, more preferably more than 92%.

In some embodiments, the measuring device is characterized in that the prisms, the lenses and the cuvette have, with a material thickness of 2 mm, a transmission of infrared beams, preferably in the wavelength range of 3910+/−100 nm and 4260+/−100 nm, of more than 85%, preferably more than 90%, more preferably more than 92%.

In some embodiments, the measuring device is characterized in that the plastic is to more than 90% by weight a polysulfone, a polyethersulfone, a polymethyl methacrylate, a polycarbonate, a polytetrafluoroethylene (Teflon), polyethersulfone, poly(arylene sulfone), a polyimide, a polyamide and/or a mixture of at least one of the polymers listed and optionally a further polymer.

In some embodiments, the measuring device is characterized in that the at least one prism of the sensor unit is produced from a polysulfone and/or a polyethersulfone and/or a polycarbonate.

In some embodiments, the measuring device is characterized in that the lenses are produced from a polysulfone and/or a polyethersulfone and/or a polycarbonate.

In some embodiments, the measuring device is characterized in that at least the lenses and the at least one prism of the sensor unit for determining the CO2 concentration, as well as the cuvette, are produced from a polysulfone and/or a polyethersulfone and/or a polycarbonate.

In some embodiments, the measuring device is characterized in that a surface of one of the prisms together with a surface of a second prism form a tapering gap, the cuvette having an outer cross section matching the tapering gap in the region of the sensor connector for attaching the sensor unit for determining the CO2 concentration.

In some embodiments, the measuring device is characterized in that the cuvette has an interior, the interior having at least two side faces which run parallel to one another, the two side faces respectively defining a plane (C), and the cuvette having at least two outer faces, the outer faces respectively defining a plane (B, E) and the two planes (B, E) being at an angle (A) with respect to one another, and the planes (C) being at an angle (D) to the planes (B, E), the angle (D) being half as great as the angle (A).

In some embodiments, the measuring device is characterized in that the cuvette comprises a coupling, the coupling being configured and adapted to connect a Y-piece and/or an expiratory device to the cuvette in such a way as to convey gas.

In some embodiments, the measuring device is characterized in that the cuvette and the Y-piece and/or the expiratory device are connected to one another rotatably.

In some embodiments, the measuring device is characterized in that the cuvette and the Y-piece and/or the expiratory device are connected so that they are secured against accidental release.

In some embodiments, the measuring device is characterized in that the coupling for connecting the cuvette and the Y-piece and/or the expiratory device has at least one seal, at least one retaining ring and at least one axial bearing, the retaining ring securing against accidental release.

In some embodiments, the measuring device is characterized in that at least one axial bearing and one retaining ring are formed together as a functional component.

In some embodiments, the measuring device is characterized in that the cuvette has a connector for a gas-conveying connection to a patient interface, this connector being produced in one piece with the cuvette.

In some embodiments, the measuring device is characterized in that the cuvette and the measuring unit are releasably connected to one another by means of at least one connecting element of the measuring unit and at least one connecting element of the cuvette.

In some embodiments, the measuring device is characterized in that the connecting elements form a click system.

In some embodiments, the measuring device is characterized in that the sensor connector for attaching a sensor unit for determining the respiratory gas flow rate has at least one feed-through for at least one sensor pin.

In some embodiments, the measuring device is characterized in that the sensor connector has at least one socket for receiving the sensor unit for determining the respiratory gas flow rate, the at least one feed-through for the at least one sensor pin being arranged in the socket.

In some embodiments, the measuring device is characterized in that the sensor connector for attaching a sensor unit for determining the CO2 concentration is configured and adapted to receive the cuvette so that the outer faces of the cuvette bear with a form-fit on the prisms and/or the lens.

In some embodiments, the measuring device is characterized in that the sensor connector for attaching a sensor unit for determining the CO2 concentration is configured substantially as two opposite planar smooth outer faces of the side walls of the cuvette.

In some embodiments, the measuring device is characterized in that the sensor connector for attaching a sensor unit for determining the respiratory gas pressure has at least one recess for receiving the sensor head of the sensor unit as well as a bore from the recess into the interior of the cuvette.

In some embodiments, the measuring device is characterized in that the cuvette has an overall length of less than 120 mm, preferably less than 80 mm. If an external pressure measurement is provided, in some embodiments the length inclusive of the Y-piece may be less than 75 mm, in some embodiments less than 65 mm. In the case of an internal pressure measurement, the length inclusive of the Y-piece may be less than 70 mm, in some embodiments less than 55 mm.

In some embodiments, the measuring device is characterized in that the wall thickness of the cuvette in the region of the sensor connector for attaching a sensor unit for determining the CO2 concentration is between 0.5 mm and 3 mm, preferably between 0.8 mm and 2 mm.

In some embodiments, the measuring device is characterized in that the wall thickness of the cuvette in the region of the sensor connector for attaching a sensor unit for determining the CO2 concentration has a tapering cross-sectional profile.

In some embodiments, the measuring device is characterized in that the cuvette can be integrated into a patient interface.

In some embodiments, the measuring device is characterized in that the at least two sensor units of the measuring unit are arranged together in a housing.

In some embodiments, the measuring device is characterized in that at least three sensor units (41, 42, 43) of the measuring unit are arranged in the one housing.

In some embodiments, the measuring device is characterized in that the sensor unit for determining the respiratory gas flow rate operates according to the principle of a thermal gas flow rate determination.

In some embodiments, the measuring device is characterized in that the sensor unit for determining the respiratory gas flow rate operates according to the principle of thermal anemometry, preferably hot wire anemometry.

In some embodiments, the measuring device is characterized in that the sensor unit for determining the respiratory gas flow rate comprises at least one sensor pin, the at least one sensor pin being fed through the feed-through at least partially into the interior of the cuvette.

In some embodiments, the measuring device is characterized in that the sensor unit for determining the respiratory gas pressure determines the respiratory gas pressure by means of a difference from atmospheric pressure.

In some embodiments, the measuring device is characterized in that the sensor unit comprises at least one sensor head.

In some embodiments, the measuring device is characterized in that the cuvette has a weight of less than 80 g, preferably less than 30 g.

In some embodiments, the measuring device is characterized in that the measuring device, consisting of the measuring unit and the cuvette, has an overall weight of less than 120 g, preferably less than 60 g.

In some embodiments, the measuring device is characterized in that the measuring unit is configured as a multi-use article and is reusable and/or the cuvette 5 is configured as a single-use article.

The object is furthermore achieved by a system for analyzing a respiratory gas flow, at least comprising
a ventilator apparatus,
a patient interface,
   wherein the system furthermore comprises a measuring device as described above, the measuring device being connected to the patient interface and the ventilator apparatus in such a way as to convey gas.

In some embodiments, the system is characterized in that the cuvette is connected to the patient interface by means of the connector and the cuvette is connected to the ventilator apparatus by means of the Y-piece.

In some embodiments, the system is characterized in that at least the measuring unit of the measuring device is arranged in and/or on the ventilator apparatus.

The object is furthermore achieved by a cuvette for use in a measuring device for analyzing a respiratory gas flow, wherein the cuvette comprises at least two sensor connectors for attaching sensor units for determining at least one respiratory gas flow rate and at least one CO2 concentration of a respiratory gas.

The object is furthermore achieved by a measuring unit for use in a measuring device for analyzing a respiratory gas flow, wherein the measuring unit comprises at least two sensor units, at least one sensor unit being configured to determine a respiratory gas flow rate and at least one sensor unit being configured to determine a CO2 concentration in a respiratory gas.

The object is furthermore achieved by a sensor unit for determining the CO2 concentration of a respiratory gas, wherein the sensor unit comprises at least one beam source, at least one detector unit, at least one mirror, at least two lenses and at least one prism.

In some embodiments, the sensor unit is characterized in that at least two of the lenses are configured as Fresnel lenses and at least one of the two lenses is arranged on the at least one prism.

In some embodiments, the sensor unit is characterized in that the at least one prism is materially connected to at least one of the at least two lenses.

In some embodiments, the sensor unit is characterized in that the at least one prism is produced in one piece with at least one of the at least two lenses.

In some embodiments, the sensor unit is characterized in that the at least one prism and the lenses are produced from a plastic, the plastic having, with a material thickness of 2 mm, a transmission of infrared beams, particularly in the wavelength range of 3910+/−100 nm and 4260+/−100 nm, of more than 85%, preferably more than 90%, more preferably more than 92%.

In some embodiments, the sensor unit is characterized in that the plastic is to more than 90 wt % a polysulfone, a polyethersulfone, a polymethyl methacrylate, a polycarbonate, a polytetrafluoroethylene (Teflon), polyethersulfone, poly(arylene sulfone), a polyimide, a polyamide and/or a mixture of at least one of the polymers listed and any one or more desired further polymers.

The object is furthermore achieved by a prism for use in a sensor unit as described above, wherein the prism is produced in one piece with a lens, the lens being a Fresnel lens.

The object is furthermore achieved by a method for producing optical components from polysulfone and/or polyethersulfone and/or polycarbonate by injection molding, wherein the optical components are prisms and lenses.

In some embodiments of the method, the prism is produced in one piece with a lens.

It should be pointed out that the features mentioned individually in the claims may be combined with one another in any desired technically expedient way and represent further configurations of the invention. The description additionally characterizes and specifies the invention particularly in connection with the figures.

It is furthermore pointed out that a conjunction “and/or” used herein, standing between two features and linking them, is always to be interpreted as meaning that in a first configuration of the subject matter according to the invention only the first feature may be present, in a second configuration only the second feature may be present, and in a third configuration both the first and the second feature may be present.

A ventilator apparatus is intended to mean any apparatus that assists a user or patient with natural respiration, takes over the ventilation of the user or living being (for example a patient and/or neonate and/or preterm) and/or is used for respiratory therapy and/or influences the respiration of the user or patient in another way. This includes, for example but not exclusively, CPAP and BiLevel apparatuses, narcotic or anesthetic apparatuses, respiratory therapy apparatuses, (clinical, out-of-clinic or emergency) ventilator apparatuses, high-flow therapy apparatuses and cough machines. Ventilator apparatuses may also be understood as diagnostic apparatuses for ventilation. Diagnostic apparatuses may in this case generally be used to record medical and/or breathing-related parameters of a living being. This also includes apparatuses that can record and optionally process medical parameters of patients in combination with the respiration or exclusively relating to respiration.

A patient interface may, unless otherwise expressly described, be understood as any peripheral apparatus that is intended for interaction, in particular for therapeutic or diagnostic purposes, of the measuring device with a living being. In particular, a patient interface may be understood as a mask of a ventilator apparatus or a mask connected to the ventilator apparatus. In the scope of the invention, it is also possible to arrange the cuvette according to the invention, or the measuring device, between the mask and the ventilator apparatus so that the mask is connected to the ventilator apparatus by means of the cuvette. This mask may be a full-face mask, that is to say covering the nose and mouth, or a nasal mask, that is to say a mask covering only the nose. Tracheal tubes or cannulas and so-called nosepieces may also be used as a mask, or patient interface. In some cases, the patient interface may also be a simple mouthpiece, for example a tube, through which the living being at least exhales and/or inhales. A connection to a ventilator apparatus is not necessary in all embodiments for the measuring device according to the invention.

The measuring device according to the invention not only is suitable in particular for use in the field of the therapy and ventilation of patients, but may furthermore also be used in other fields in which an analysis of natural respiration may be desired, for example for divers, mountaineers, in protective equipment for firefighters, etc. The measuring device according to the invention may also be used in the field of determining various physiological parameters of a living being—not only with a view to diagnosis.

The measuring device according to the invention combines at least one sensor unit for determining a respiratory flow rate or a sensor unit for determining the respiratory gas pressure together with at least one sensor unit for determining the CO2 concentration in a shared housing. Correspondingly, the cuvette of the measuring device is also configured so that it has at least two corresponding sensor connectors. Jointly integrated into the cuvette, for example, there is in addition a connector for a patient interface, although the cuvette may in principle also be integratable into a patient interface. In some embodiments of the measuring device, three sensor units—respectively for determining respiratory gas flow rate, respiratory gas pressure and CO2 concentration—are also installed in the measuring unit. Preferably, the measuring device is configured so that no bypass line is needed for the measurement of respiratory gas pressure and/or respiratory gas flow rate, but instead the measurements can be carried out directly in the cuvette.

The sensor unit for determining the CO2 concentration in this case comprises optical component parts, in particular prisms and lenses, which are manufactured from a polymer that transmits IR radiation. Ideally, the cuvette is also manufactured from such a material. Manufacture from a polymer also allows simple production of the prisms in one piece with the lenses.

The measuring device may be used both with a ventilator apparatus and without a ventilator apparatus. For use of the measuring device with a ventilator apparatus, the cuvette additionally has a coupling by means of which, for example, an expiratory device (for example an expiratory valve controlled by the ventilator apparatus or a leak system) may be attached for a 1-hose configuration or a Y-piece may be attached for a 2-hose configuration. The coupling is in this case preferably designed so that no undesired leaking occurs and the cuvette and the attached Y-piece, or the expiratory device, can easily be rotated relative to one another.

Depending on the living being on/with/by which the measuring device is intended to be used, certain modifications or adaptations are to be taken into account, particularly in the cross sections of the component parts that convey gas, such as the cuvette. While for example preterms have a small tidal volume and lung volume and quite low respiratory pressures and flow rates, the smallest possible embodiment (small cross sections) with low dead space volumes are desirable in that respect. For larger living beings, for example adult humans, the small cross section may however lead to a high respiratory resistance and the cuvette must therefore be configured to be correspondingly larger than for preterms.

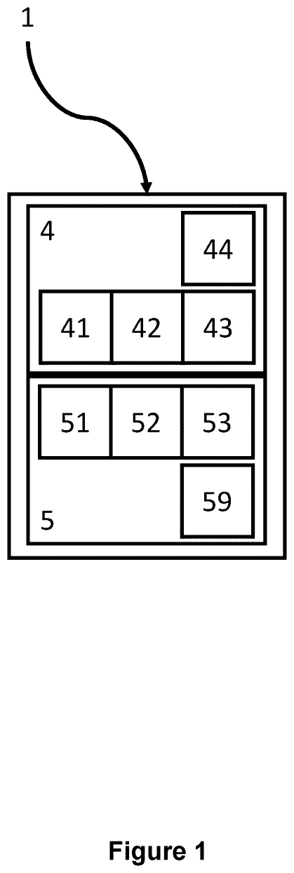

The invention is explained in more detail by way of example with the aid of FIGS. 1 to 16.

FIG. 1: schematic representation of the measuring device 1

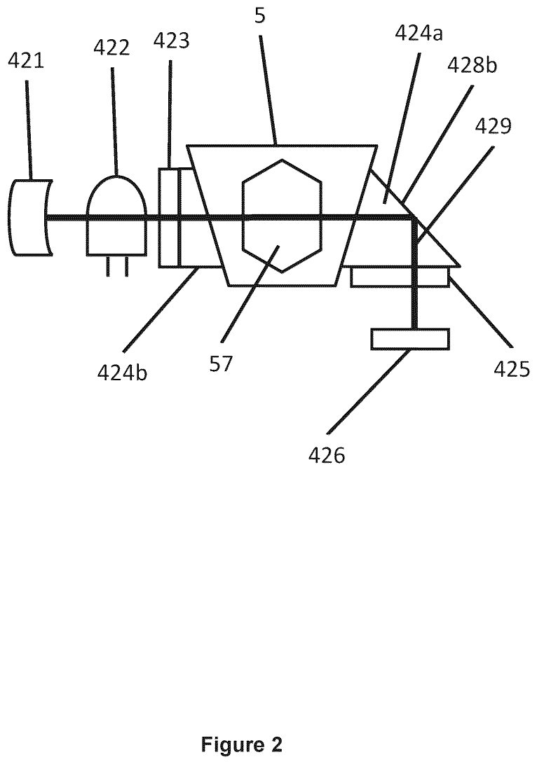

FIG. 2: cross section through the cuvette 5 and the optical components of the sensor unit 42

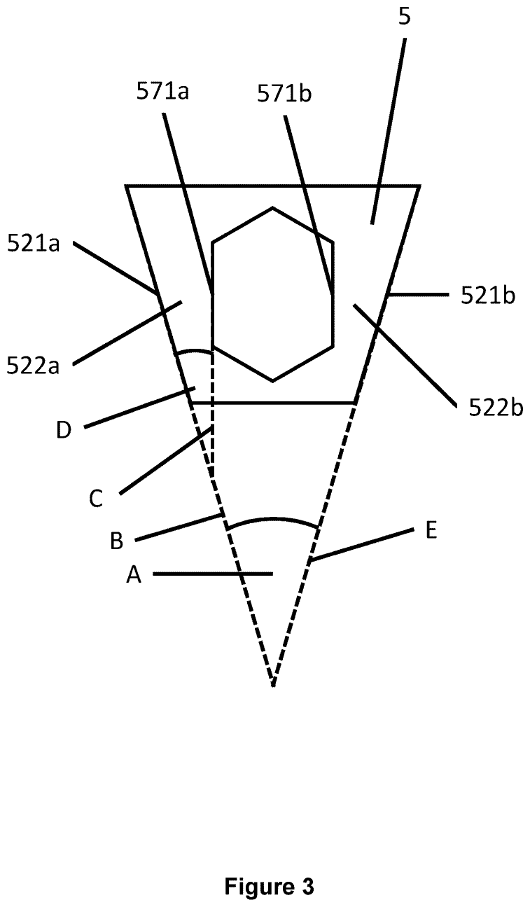

FIG. 3: cross section of the cuvette 5

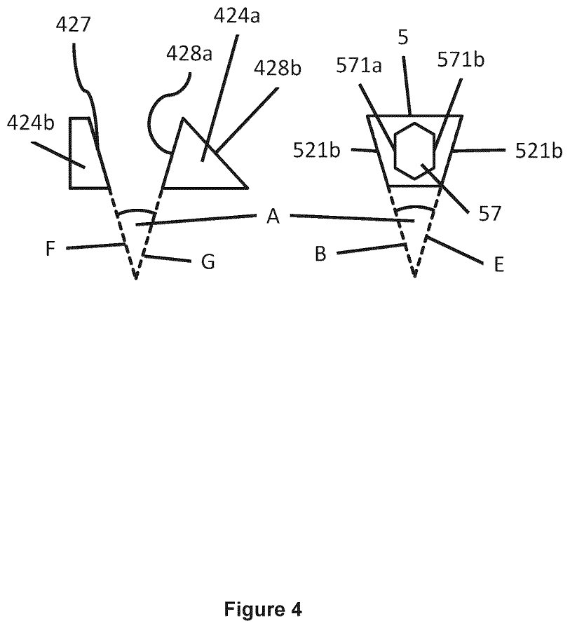

FIG. 4: cross section of the prisms 424a, 424b and the cuvette 5

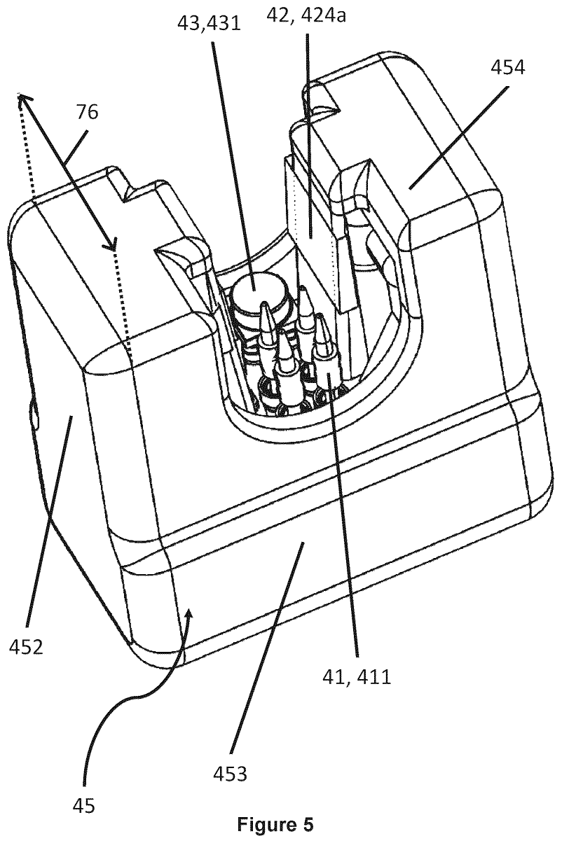

FIG. 5: perspective view of the measuring unit 4 with a housing 453

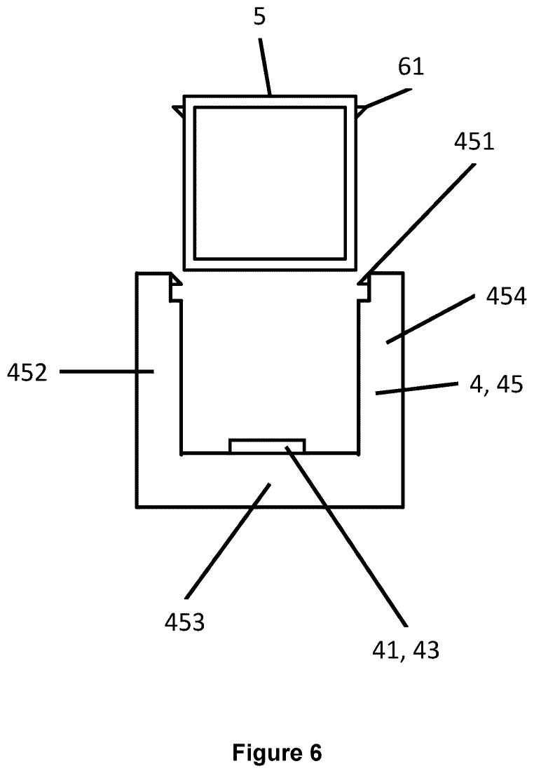

FIG. 6: connecting elements of the measuring unit 4 and the cuvette 5

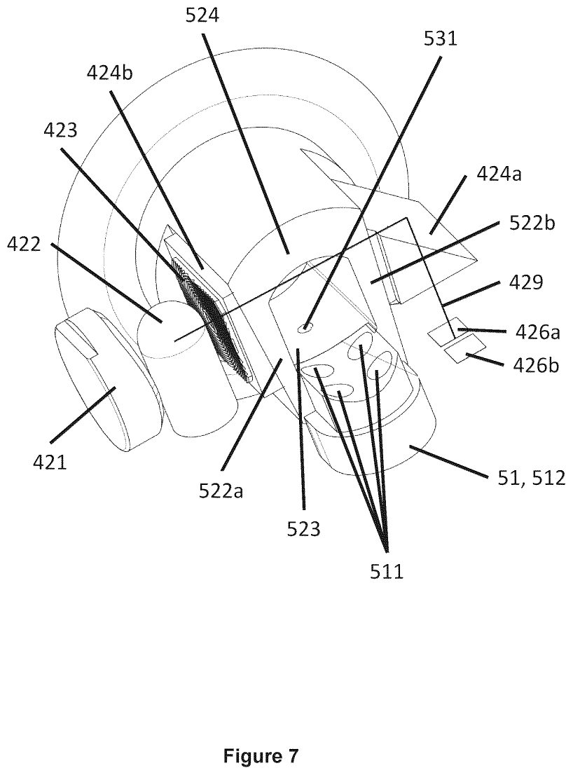

FIG. 7: perspective section through the cuvette 5 with optical components of the sensor unit 42

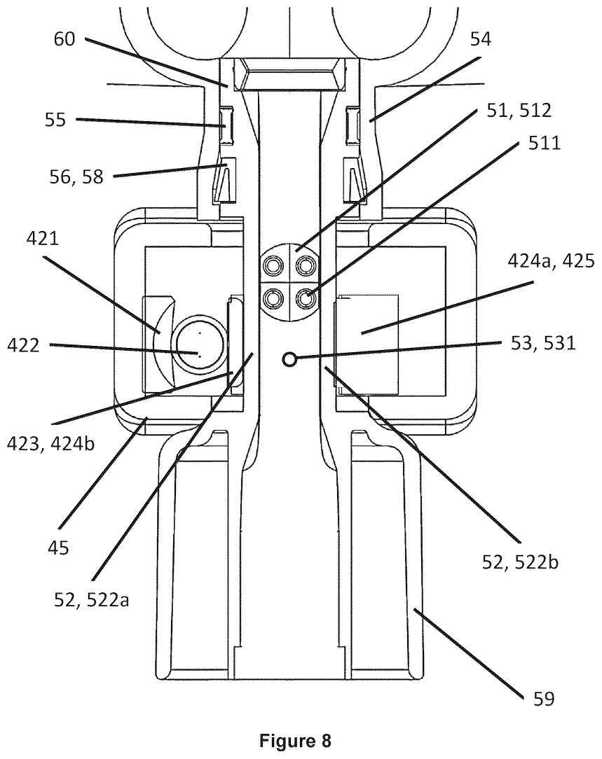

FIG. 8: cross section along the measuring device 1 in a plan view

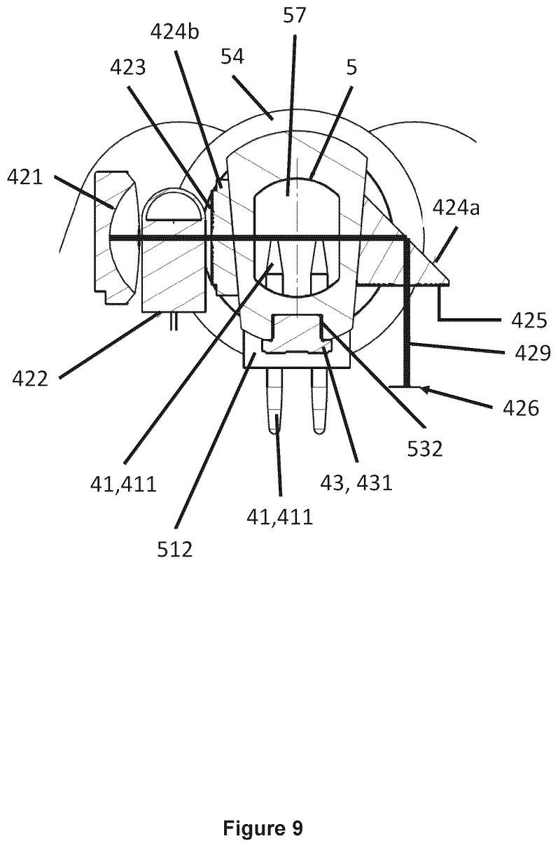

FIG. 9: cross section through the sensor unit 42 and the cuvette 5

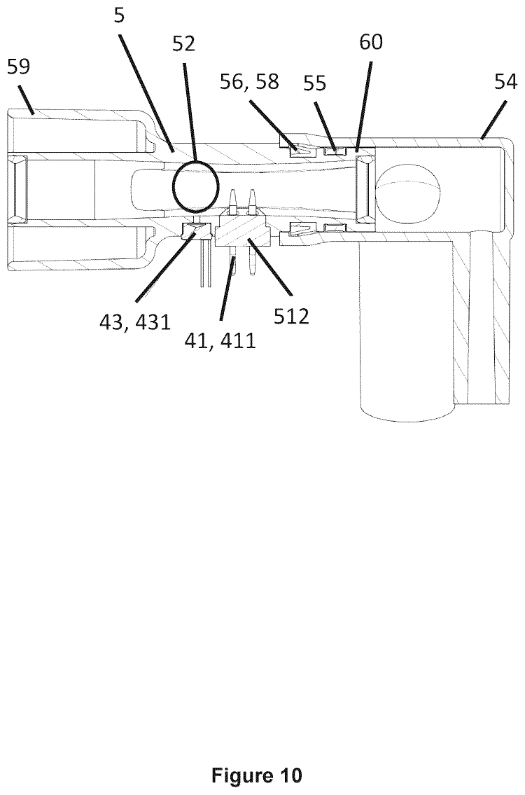

FIG. 10: cross section along the measuring device 1 through the sensor units 41, 43

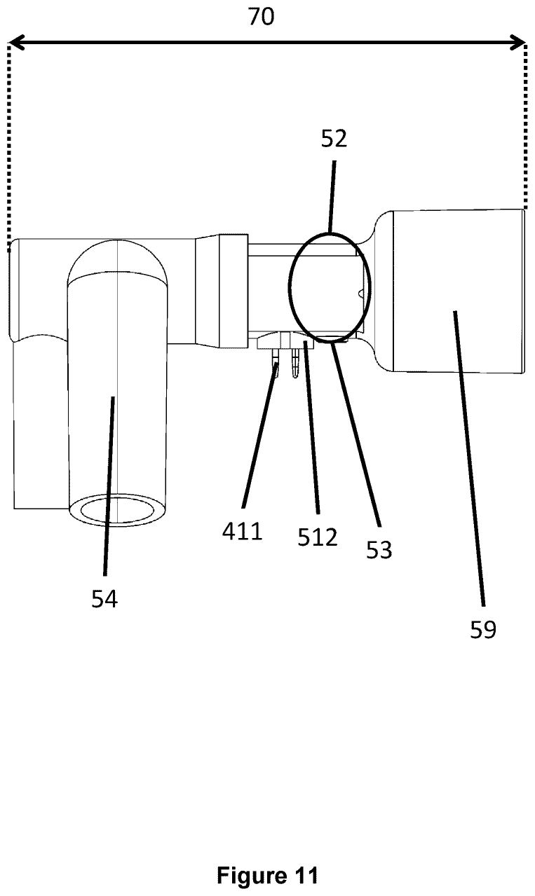

FIG. 11: dimensioning of the cuvette 5 with a Y-piece 54

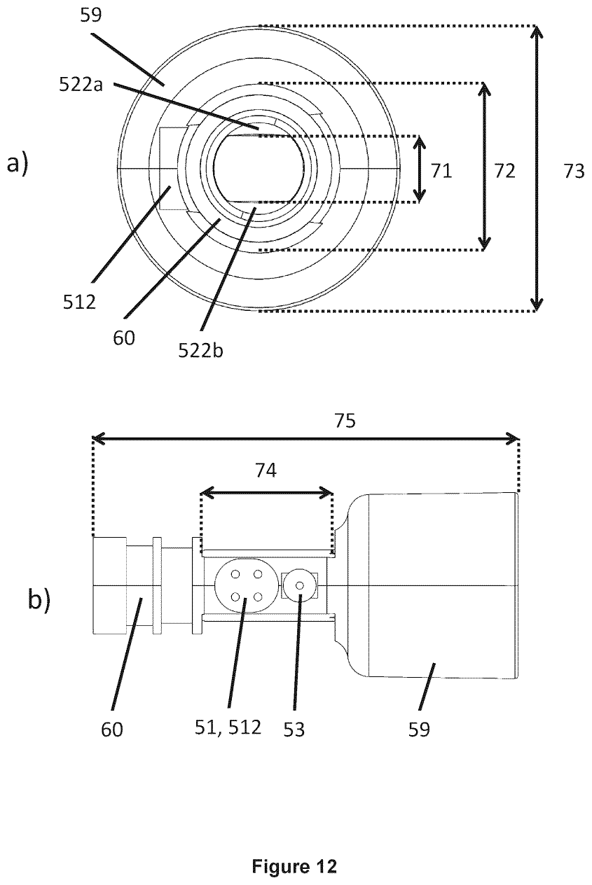

FIG. 12 a): diameter of the cuvette 5

FIG. 12 b): dimensioning of the cuvette 5 without a Y-piece 54

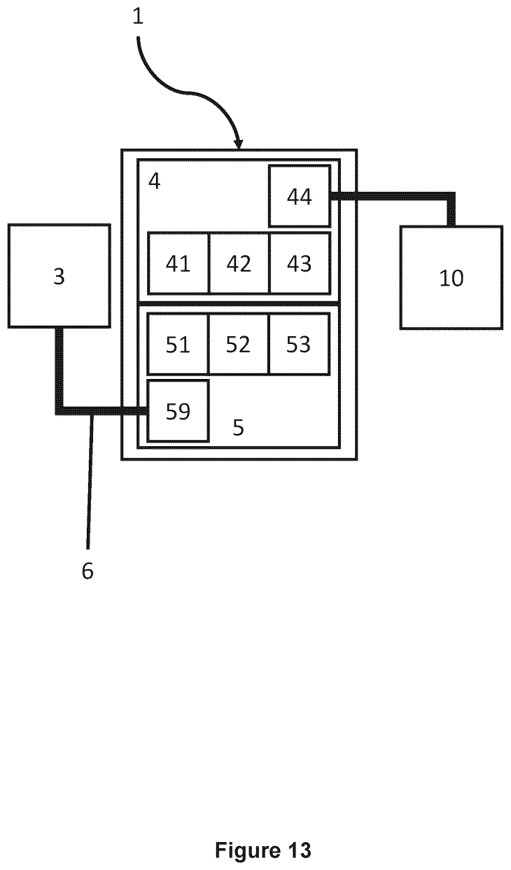

FIG. 13: schematic representation of the measuring device 1 with a patient interface 3

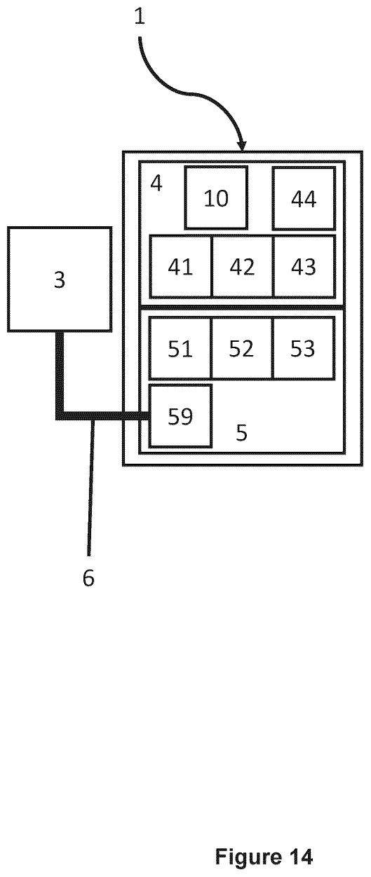

FIG. 14: schematic representation of the measuring device 1 with a patient interface 3

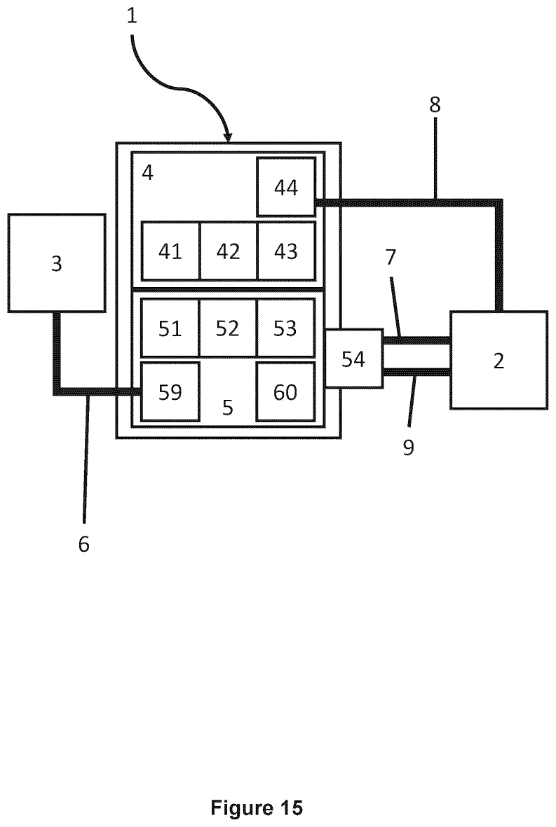

FIG. 15: schematic representation of the measuring device 1 with a patient interface 3 and a ventilator apparatus 2 in a 2-hose configuration

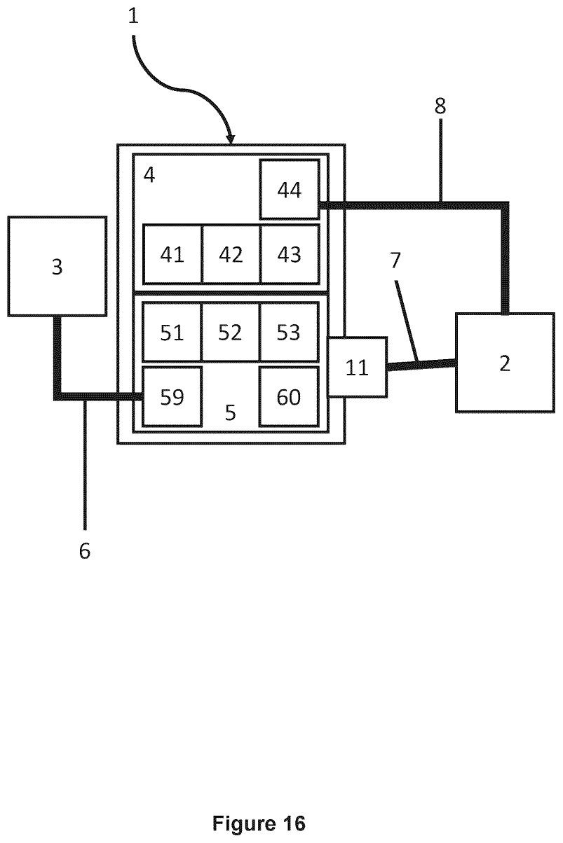

FIG. 16: schematic representation of the measuring device 1 with a patient interface 3 and a ventilator apparatus 2 in a 1-hose configuration

FIG. 1 schematically represents an exemplary embodiment of the measuring device 1 according to the invention for analyzing a respiratory gas flow. The embodiment shown comprises a measuring unit 4 and a cuvette 5 matched thereto. The measuring unit 4 comprises by way of example the three sensor units 41, 42, 43. Complementarily therewith, the cuvette 5 has three sensor connectors 51, 52, 53 for attaching the sensor units 41, 42, 43. The measuring unit 4 additionally has at least one interface 44, by means of which the measurement data and/or electrical signals generated by the sensor units 41, 42, 43 can be forwarded and/or transmitted to a corresponding signal processing unit (not represented). The cuvette 5 also has a connector 59, which may for example be connected to a patient interface 3 in such a way as to convey gas. In some embodiments, the cuvette 5 may also be integrated into the patient interface 3. In this case, the cuvette 5 is for example attached to the mask body of a patient interface 3 instead of a hose connector. In this way, for example, the connector 59 forms the transition from the patient interface 3 to the cuvette 5.

For example, the interface 44 is configured as a combined interface which forwards the signals of the sensor units 41, 42, 43 in a bundle, although it is in addition also possible for each sensor unit to have its own interface for transmitting the signals to a signal processing unit. The signal processing unit is used for example to convert the electrical signals generated by the sensor units 41, 42, 43 into measurement values and/or data, which may then be interpreted, displayed and/or further processed by corresponding conditioning, output, evaluation and/or calculation units, and possibly display elements. In some embodiments of the measuring unit 4, the signal processing unit is integrated into the measuring unit 4. Alternatively or in addition, the signal processing unit may be provided as an external apparatus, for example together with further conditioning, evaluation and/or calculation units and if appropriate also display and user interaction elements. Besides a signal processing unit, the measuring unit 4 may furthermore also be adapted and configured to display, interpret and/or further process the values and/or data output by the signal processing unit, for example by conditioning, evaluation and/or calculation units integrated in the measuring unit. For example, a display which can display and/or output the measurement values of the sensor units 41, 42, 43 may be arranged on the measuring unit 4, optionally together with user interaction elements and/or as a touchscreen.

The sensor unit 41 is for example configured as a sensor unit for determining the respiratory gas flow rate (“flow rate”), that is to say the value of the flow rate (volume per unit time) of the respiratory gas flow. The sensor connector 51 for attaching such a sensor unit 41 is configured complementarily therewith. The sensor unit 41 is, for example, a flow rate sensor which is based on a thermal measurement principle. Such a measurement principle is, for example, thermal anemometry. One exemplary embodiment of the sensor unit 41 is a hot wire anemometer. For this purpose, at least one sensor pin 411 is placed in the respiratory gas flow, this sensor pin 411 having a thin metal wire which is clamped between two metal tips, or is welded or soldered thereto. At this point, it should be pointed out that other alternative methods and sensor units for measuring the respiratory gas flow rate may be installed in the measuring unit 4. For example, no bypass line is needed for these alternative methods of measuring the respiratory gas flow rate, and it may instead be measured directly in the cuvette 5.

The sensor unit 42 is for example configured and adapted as a sensor unit for determining the CO2 concentration of the respiratory gas. In some exemplary embodiments, the sensor unit 42 is an infrared sensor unit which measures the CO2 concentration with the aid of the infrared radiation absorbed by the respiratory gas flowing through the cuvette 5. The matching sensor connector 52 of the cuvette 5 is accordingly configured so that the cuvette 5 is transparent for the infrared radiation at least in the region of the sensor connector 52.

The sensor unit 43 is for example configured as a sensor unit for determining the respiratory gas pressure in the cuvette 5. The sensor connector 53 of the cuvette 5 is configured complementarily therewith so that it can receive the sensor unit 43 and is in connection with the interior 57 of the cuvette 5. For example, this connection between the sensor unit 43 and the interior 57 of the cuvette 5 is formed by a bore 531 in the region of the sensor connector 53. For example, the respiratory gas pressure is measured by the sensor unit 43 by means of a difference from atmospheric pressure, or the ambient air pressure. For this purpose, the sensor unit 43 has for example at least one sensor head 431.

FIG. 2 shows by way of example the essential elements of the sensor unit 42 for determining the CO2 concentration of the respiratory gas in conjunction with the cuvette 5. The determination of the CO2 concentration is based by way of example on the absorption of infrared radiation by the CO2 molecules in the respiratory gas which flows through the interior 57 of the cuvette 5.

In order to generate the infrared radiation, the sensor unit 42 comprises at least one the beam source 422, for example an infrared radiator and/or an incandescent lamp. In general, beam sources emit the radiation substantially undirected and omnidirectionally. In order to be able to use as much as possible of the radiation generated by the beam source 422 for analyzing the respiratory gas flow in the cuvette 5, the sensor unit 42 comprises a mirror 421 which is adapted to reflect the radiation generated by the beam source 422 substantially in the direction of the cuvette 5. For this purpose, the mirror 421 is configured for example as a concave mirror. In some embodiments, the mirror 421 is in addition aspherical and/or anamorphic, so that better adaptation of the shape of the beam path 429 is possible. Furthermore, the mirror 421 is coated for high reflection, for example with gold. Besides gold, other coatings may also be envisioned for the mirror 421, so long as they ensure reflection of the radiation generated by the beam source 422. In some embodiments, the beam source 422 itself may also have a reflective coating so that the radiation generated by the beam source 422 is already reflected in the direction of the cuvette 5 by the beam source 422. Alternatively, the use of a directed beam source 422 may be envisioned, for example an (infrared) laser.

After the mirror 421 and the beam source 422, a lens 423 is arranged in the beam path 429. The lens 423 is for example configured as a planoconcave lens, which is used inter alia for beam development before the cuvette 5. The lens 423 is furthermore configured and adapted to act as a beam splitter. For example, the lens 423 is configured as a Fresnel lens, which allows a compact construction of the sensor unit 42. A prism 424b is arranged directly on the lens 423. In some embodiments of the cuvette 5, the outer faces 521a of the cuvette 5 are chamfered, as may be seen particularly in FIGS. 3 and 4, which leads to a prismatic effect of the cuvette 5. The prism 424b is chamfered by the same amounts, so that overall no refraction faces or refraction edges occur between the lens 423 and the outer face 521a of the cuvette 5. In some embodiments of the sensor unit 42, the lens 423 and the prism 424b are manufactured in one piece and from the same material. For example, the lens 423 and the prism 424b may be produced in one piece from a plastic, for example a polysulfone, a polyethersulfone and/or a polycarbonate, by an injection molding method. According to some embodiments, the material may also be Teflon. What is crucial for the selection of the plastic is a high transmission of the infrared radiation. For example, with a material thickness of 2 mm, the plastic should have a transmission of more 85% for certain individual wavelengths in the range of between 3600 nm and 4550 nm, preferably in the wavelength range of 3910+/−100 nm and 4260+/−100 nm. In some embodiments, a transmission of more than 90% or more than 92% is preferable. The high transmission is necessary in particular for the wavelengths in the range of from 4200 nm to 4400 nm and 3800 nm to 4000 nm, preferably in the wavelength range of 3910+/−100 nm and 4260+/−100 nm.

The use of a plastic/polymer for the cuvette 5 and essential parts (for example the prisms 424a, 424b, lenses, etc.) of the measuring unit 4 makes it possible for the measuring unit overall to have a very low weight. For example, the cuvette 5 and the measuring unit 4 together weigh less than 120 g, preferably less than 60 g, more preferably less than 30 g. Both the cuvette 5 and the measuring unit 4 each weigh for example less than 80 g, preferably less than 30 g, more preferably less than 15 g.

In the interior 57 of the cuvette 5, the infrared radiation encounters the respiratory gas flow, parts of the infrared radiation being absorbed by CO2 molecules that are contained in the respiratory gas. In this case, in particular, the wavelength range around 4260 (+/−10) nm is absorbed, the wavelength range around 3910 (+/−10) nm substantially not being absorbed by the CO2 molecules or other (regular) constituents of the respiratory gas of a living being. The part of the infrared radiation that is not absorbed by the respiratory gas then passes through the side face 571b and the side wall 522b of the cuvette 5 and emerges from the outer face 521b. The prism 424a is arranged bearing with a form-fit on the outer face 521b. The infrared radiation enters the prism 424a through the surface 428a of the prism 424a. The prism 424a is for example configured as an inverting prism, preferably as a roof prism. In addition, the prism 424a is aligned and configured so that the infrared beams strike the face 428b of the prism 424a for example at an angle of at least 45° or more. The infrared beams are totally reflected at the face 428b in the direction of the lens 425, which is arranged on the prism 424a. The lens 425 is by way of example configured as a Fresnel lens, and in some embodiments is for example produced in one piece with the prism 424a, so that the prism 424a merges materially into the lens 425. The prism 424a and the lens 425 may for example be produced by injection molding and be manufactured simultaneously in the same tool.

The lens 425 is in addition adapted and configured so that a uniform image is projected onto the detector unit 426. The detector unit 426, which has for example two detector faces 426a, 426b, converts the incident infrared radiation into electrical signals. The detector face 426a is for example configured to detect infrared radiation in the wavelength range around 4260 (+/−100) nm, and the detector face 426b is for example configured to detect infrared radiation in the wavelength range around 3910 (+/−100) nm. By comparing the two radiation intensities detected by the detector unit 426, it is possible to ascertain how much of the infrared radiation in the wavelength range around 4260 (+/−100) nm is absorbed by the CO2 of the respiratory gas. It is thus important for the two detector faces 426a, 426b to be irradiated identically. The detector face 426b for detecting infrared radiation in the wavelength range around 3910 (+/−100) nm is therefore used as a reference detector, while the detector face 426a for detecting infrared radiation in the wavelength range around 4260 (+/−100) nm may be regarded as a measuring detector. The arrangement of the prisms 424a, 424b and the lenses 423, 425 serves for beam guiding and beam development, so that the same image can be projected onto both detector faces 426a, 426b, that is to say the beams are split equally. In some embodiments, the detector unit 426 comprises more than two detector faces, for example in order to measure further gas constituents of the respiratory gas flow. For example, further detector faces for determining the O2 concentration (for example in the range around 1580 nm and/or 1270 nm) and/or the CO concentration (for example in the range around 4670 nm and/or 2340 nm) of the respiratory gas may be installed as part of the detector unit 426.

The individual constituents of the sensor unit 42 and of the sensor connector 52, or of the cuvette 5, in particular lenses, prisms and the cuvette 5 itself, are for example manufactured from a plastic that is transparent for infrared beams. Examples of such plastics are polysulfone, polyethersulfone, polymethyl methacrylate, polycarbonate, polytetrafluoroethylene, polyethersulfone, poly(arylene sulfone), polyimide, polyamide. In some embodiments, a mixture of the aforementioned plastics or a mixture of one and/or a plurality of the plastics with further plastics may also be envisioned. In the case of a mixture of plastics, the aforementioned plastic or plastics should make up more than 90% by weight of the plastics mixture. For example, a plastics mixture may consist of 46% polysulfone, 46% polyethersulfone and 8% polystyrene. Plastics and plastics mixtures which, with a material thickness of 2 mm, have a transmission of infrared radiation, particularly in the wavelength range of from 3600 nm to 4550 nm, preferably in the wavelength range of 3910+/−100 nm and 4260+/−100 nm, especially in the range of from 3850 nm to 3960 nm and 4200 nm to 4300 nm, of more than 85%, preferably more than 90%, are preferred. In some embodiments, a transmission of more than 92% is desirable. Especially polysulfones, polyethersulfones and/or polycarbonates are suitable as material for the lenses 423, 425, the prisms 424a, 424b and the cuvette 5.

While in many exemplary embodiments the lenses, the prisms and the cuvette 5 are manufactured from the same material, it is also conceivable for the materials of the individual constituents to vary. In some embodiments, for example, the lenses and/or the prisms may be manufactured from a mineral, or an inorganic material, instead of a plastic.

A further aspect, which for example distinguishes the material selection of the cuvette 5, the prisms and the lenses, is that the entire optical arrangement (mirror, beam source, lenses, prisms, cuvette) functions without the use of an antireflection coating.

FIG. 3 shows an exemplary embodiment of the cuvette 5 partially as a cross section in the region of the sensor connector 52 for attaching the sensor unit 42 for determining the CO2 concentration in the respiratory gas flow. The outer faces 521a, 521b of the cuvette 5 run for example in a chamfered fashion, so that they converge with one another in the shape of a wedge. The outer faces 521a, 521b are for example planar, so that the outer faces 521a, 521b respectively define planes B and E, which intersect at an angle A. For beam guiding, the interior 57 of the cuvette 5 has, at least in the region of the sensor connector 52, two plane-parallel inner faces 571a, 571b, which define the planes C. The planes C in this case respectively intersect the planes B and E at an angle D which, for example, is half the angle A. In some embodiments, the outer faces 521a, 521b are not chamfered, so that the planes B, E and C never intersect, and the outer faces 521a, 521b thus run plane-parallel to the inner faces 571a, 571b, which likewise run plane-parallel to one another. If the prisms 423, 425 are matched in their arrangement and the course of the surfaces 427, 428a to the course of the outer faces 521a, 521b, the infrared radiation for example enters the interior 57 of the cuvette 5 from the inner face 521a approximately at an angle of 90° and then strikes the inner face 521b at an angle of about 90°. In some embodiments, the radiation does not exactly run axially parallel and the radiation is, for example, already concentrated here in the direction of the detectors. It may therefore happen that the radiation runs, or strikes the aforementioned faces, only approximately at an angle of 90° and deviations from the angle of 90° occur in this case. The corresponding course of the beam is, for example, developed by the lens 423 so that the beams run perpendicularly to the inner faces 571a, 572b.

FIG. 4 schematically represents the prisms 424b, 424a and the cuvette 5 in order to illustrate the course of the surfaces 427, 428a of the sensor unit 42 in relation to the outer faces 521a, 521b of the sensor connector 52 of the cuvette 5. For example, not only the surfaces 427 and 428a but also the outer faces 521a, 521b are chamfered toward one another in the shape of a wedge. Various planes B, E, F, G are defined by the faces 427, 428a, 521a and 521b: the surface 427 defines the plane F, the surface 428a defines the plane G, the outer face 521a defines the plane B and the outer face 521b defines the plane E. The faces 427, 428a, 521a, 521b are configured so that the planes F and B run parallel to one another and the planes G and E run parallel to one another. The planes F and B are in this case at an angle A with respect to the planes G and E. As already described, in some embodiments the faces 427, 428a, 521a, 521b are not chamfered but are all plane-parallel to one another, which correspondingly also applies for the planes B, E, F, G. It may for example be advantageous for the faces 427, 428a, 521a, 521b at least slightly to be chamfered and converge in the shape of a wedge so that simpler positioning of the cuvette 5 in the sensor unit 42 may be achieved, and form-fit bearing of the outer faces 521a, 521b on the respective surfaces 427 or 428a may also be ensured.

Advantageously, the three sensor units 41, 42, 43 are arranged together in a housing 45, as is represented by way of example in a perspective view in FIG. 5. The housing is for example configured roughly in the shape of a U, so that the cuvette 5 can be inserted into the resulting intermediate space. The sensor units 41, 43 for determining the respiratory gas flow rate and the respiratory gas pressure are, for example, arranged next to one another in the housing bottom 453 of the housing 45. In order to ensure a beam path 429 through the cuvette 5 and therefore also through the respiratory gas, the optical components of the sensor unit 42 for determining the CO2 concentration of the respiratory gas are arranged in the housing sides 452, 454. For example, the beam source 422 is arranged together with the mirror 421, the lens 423 and the prism 424b in the one housing side 452, and the prism 424a is arranged with the lens 425 in the other housing side 454. The detector 426, or the detector faces 426a, 426b, are for example likewise arranged in the housing side 454 or below the housing side 454 in the housing bottom 453 or at the transition from the housing side 454 to the housing bottom 453. If the measuring unit 4 has an interface 44 for attaching external apparatuses, this interface 44 is for example also arranged in the housing bottom 453, although arrangement of the interface 44 in one of the housing sides 452, 454 may alternatively also be envisioned.

The power supply of the measuring unit 4, or of the sensor units 41, 42, 43, is produced for example by means of an optionally replaceable battery or accumulator integrated in the housing 45. In some embodiments, the measuring unit 4 is supplied with power via an interface. For example, an attached ventilator apparatus may also undertake the power supply of the measuring unit 4. For this purpose, the interface between the ventilator apparatus and the measuring unit 4 may for example be adapted so that the power supply also takes place besides the signal and/or data transmission. Alternatively or in addition, the measuring unit 4 is provided with an internal or external power supply unit, which allows a direct power supply.

For secure and releasable connection between the measuring unit 4 and the cuvette 5, for example both the measuring unit 4 and the cuvette 5 may have connecting elements 451, 61, as represented in a very simplified fashion in FIG. 6 with the aid of an exemplary embodiment. For example, the connecting elements 451, 61, may engage in one another and block one another. For example, the connecting elements 451, 61 may together form a click mechanism, so that the measuring unit 4 can be clipped onto the cuvette 5. For this purpose, it is also conceivable for a click noise to give audible feedback that the measuring unit 4 is connected to the cuvette 5.

In some embodiments, in addition or alternatively, it is for example also conceivable for the measuring unit 4 to have a kind of cover or latch, which is closed after the measuring unit 4 is connected to the cuvette 5 and which fixes the cuvette 5 in the measuring unit 4.

FIG. 7 shows the essential optical components (mirror 421, beam source 422, lens 423 with prism 424b, prism 424a with lens 425, detector faces 426a and 426b) of the sensor unit 42 and a cross section through the cuvette in the region of the sensor connector 52 for the sensor unit 42 for determining the CO2 concentration. For reasons of representation, further electronics components and the housing 45 are not represented. The feed-through openings 511 of the sensor connector 51 for the sensor unit 41 for measuring the respiratory gas flow rate and the bore 531 of the sensor connector 53 for the sensor unit 43 for measuring the respiratory gas pressure are arranged in the bottom 523 of the cuvette 5. The feed-through openings 511 are, for example, arranged in a socket 512 which is configured to receive the sensor unit 41 for determining the respiratory gas flow rate. The socket 512 is for example manufactured as a separate part and into a corresponding recess, which is adapted to hold the socket 512. For example, the socket 512 is clipped into the corresponding recess of the cuvette, for example by means of a click mechanism. Furthermore, it is also conceivable and possible for the socket 512 to be produced in one piece with the cuvette 5 and if appropriate to be characterized only by the feed-throughs 511. In the embodiment shown, the cuvette 5, or the socket 512, has four feed-throughs 511. In another exemplary embodiment, the socket 512, or the cuvette 5, may have five feed-throughs 511, a number of in total from one to ten feed-throughs 511 generally being possible. The feed-throughs 511 may for example also be distributed over a plurality of sockets 512 and/or arranged at a plurality of different positions of the cuvette 5.

Particularly when the measuring unit 4 is configured as a reusable unit and the cuvette 5 is configured as a single-use article, the above-described possibility of connecting the cuvette 5 releasably to the measuring unit 4 is crucial. If the measuring unit 4 is configured as a reusable unit, it may thus be taken into consideration here that the entire measuring unit is accessible for cleaning and, for example, can withstand sterilization and/or disinfection and is not damaged. In some embodiments, for example, certain individual parts of the measuring unit 4 may also be replaceable—for example the sensor pins 411 and/or the sensor head 431. The interior of the measuring unit 4 may, for example, also be sealed so that no substances can enter the housing 45 from the outside. It would therefore be sufficient that the surfaces of the measuring unit 4 are sterilizable and/or can be disinfected. In particular, it is in this case necessary to take care that the materials used are stable in relation to the cleaning and are not dissolved or damaged in another way.

In some embodiments, both the cuvette 5 and the measuring unit 4 may be configured as a single-use articles. In this case, the measuring unit 4 may for example be firmly connected to the cuvette 5 so that the measuring device is configured as a single workpiece, without the measuring unit 4 and the cuvette 5 being separable. Furthermore, besides the measuring unit 4, the cuvette 5 may also be configured as a multi-use article, that is to say reusably. It is in this case assumed that the cuvette 5 is for this purpose sterilizable and can also be disinfected.

In the state shown in FIG. 7, the sensor unit 42 is connected to the cuvette 5, the prisms 424a and 424b bearing with a form-fit externally on the side walls 522a and 522b. In addition, in the exemplary representation of the cuvette in FIG. 7 it may be seen that the pressure measurement by means of the sensor connector 53, or the bore 531, may carried out in the same region as the CO2 measurement by means of the sensor unit 42. For this purpose, the bore 531 is formed in the bottom 523 of the cuvette 5, while the side walls 522a and 522b are formed in the same portion of the cuvette 5 so that they function as a sensor connector 52 for the sensor unit 42 for measuring the CO2 concentration.

In principle, arrangement of the sensor connector 51 for the sensor unit 51 for determining the respiratory gas flow rate in the region of the sensor connector 52 for determining the CO2 concentration would also be possible, although it would then be necessary to take care that either no sensor pins 411 are arranged in the interior 57 of the cuvette 5 or they are placed at least outside the beam path 429 of the sensor unit 42.

FIG. 8 shows a section along an exemplary embodiment of the cuvette 5 with the measuring unit 4 in a plan view. For example, a coupling is formed on the cuvette 5, by means of which for example a Y-piece 54, an expiratory device 11 and/or in general an adapter for establishing a gas-conveying connection, for example to a ventilator apparatus, may be attached. In the embodiment represented by way of example in FIG. 8, the coupling 60 is configured so that the cuvette 5 can be connected to the Y-piece 54 in such a way as to convey gas. Fitted on the coupling 60, for connection to the Y-piece 54, there is a retaining ring 58, which at the same time also serves as an axial bearing 56. The retaining ring 58 is for example configured so that the Y-piece 54 can be fitted over the coupling 60 but then hooks on the retaining ring 58 so that simple retraction of the Y-piece 54 is no longer possible. Because the retaining ring 58 is at the same time configured as an axial bearing 56, the Y-piece 54 can be connected to the cuvette 5, or the coupling 60, while being freely rotatable. In some embodiments of the measuring device 1, or of the cuvette 5, the cuvette 5 does not comprise a coupling 60, particularly when no further apparatuses (for example a ventilator apparatus 2) are intended to be connected to the cuvette in such a way as to convey gas. The coupling 60 may in some embodiments also be configured as a simple tube piece, by means of which for example a resilient hose or an adapter can be fitted on.

In order to avoid a leak, that is to say escape of the respiratory gas, at the connection between the Y-piece 54 and the cuvette 5, a seal 55 is furthermore arranged on or around the coupling 60. The seal 55 extends, for example, around the coupling 60. For example, the seal 55 is produced from a silicone, for example with a low prestress. While the combined retaining ring 58 with an axial bearing 56 ensures the basic rotatability of the Y-piece 54, the turning resistance, that is to say the difficulty or ease of motion of the seal, is primarily determined by the contact/friction occurring between the seal 55 and the Y-piece 54. This turning resistance is in this case particularly low, that is to say the Y-piece 54 and the cuvette 5 can be rotated easily relative to one another. Preferably, the turning resistance between the Y-piece 54 and the cuvette 5 lies in a range of from 10 to 80 N*cm.

The bore 531 of the sensor connector 53 and the socket 521 of the sensor connector 51 with the feed-throughs 511, of which there are for example four, are arranged in the bottom 523 of the cuvette 5. The prisms 424a, 424b of the sensor unit 42 for determining the CO2 concentration of the respiratory gas bear externally on the side walls 522a, 522b of the cuvette 5. The beam path 429 (not represented in FIG. 8), starting from the beam source 422, runs through the interior 57 of the cuvette 5 in the region above the bore 531 of the sensor connector 53. The three sensor units 41, 42, 43 are for example arranged together in a housing 45 of the measuring unit 4, only the essential optical elements of the sensor unit 42 for determining the CO2 concentration being represented in FIG. 8. In relation to the view of FIG. 8, the sensor units 41 and 43 are arranged below the respective sensor connectors 51 and 53 in the housing 45 (as represented by way of example in FIG. 5).

The cuvette 5 is, for example, divided so that the sensor connectors 51, 52, 53 are arranged between the coupling 60 and the connector 59. The measuring unit 4 is accordingly likewise attached between the coupling 60 and the connector 59.

A further cross-sectional view through the cuvette 5 and the measuring unit 4 is represented by way of example in FIG. 9, the housing 45 and the electronics of the sensor units 41, 42, 43 mostly not being depicted for reasons of clarity. The cross section of FIG. 9 is in this case perpendicular to the cross section of FIG. 8.

The beam path 429 of the sensor unit 42 for determining the CO2 concentration of the respiratory gas starts from the beam source 422, the beam source 422 emitting beams at least both in the direction of the mirror 421 and in the direction of the lens 423. In order to achieve the greatest possible beam efficiency, the mirror 421 is configured for example as a gold-coated aspherical anamorphic concave mirror, the mirror 421 reflecting the beams coming from the beam source 422 in the direction of the lens 423. The lens 423, for example configured as a Fresnel lens, is produced in one piece with the prism 424b and is used for the beam forming and therefore the alignment of the beams with the cuvette 5. The beams are, for example, aligned so that they travel perpendicularly with respect to the direction of the respiratory gas flow through the cuvette 5. The prism 424b is for example shaped so that it has a chamfered side, which can bear with a form-fit on the side wall 522a of the cuvette 5. The cant is configured at an angle which also corresponds to the chamfer of the side wall 522a. On the opposite side of the cuvette 5 from the prism 424b, the prism 424a, which is configured for example as a roof prism, bears with a form-fit on the side wall 522b of the cuvette 5. The prism 424a is for example formed in one piece with a lens 425, this lens being configured for example as a Fresnel lens. At the face 428b of the prism 424a, the beams are totally reflected and guided in the direction of the detector 426. The lens 425 is used for example to shape the beam, so that the same beam image is respectively projected onto the two detector faces 426a, 426b. The detector faces 426a, 426b are configured so that the detector faces 426a, 426b detect two different wavelengths, one detector face being configured as a reference detector and one detector face being configured as a measuring detector.

In the embodiment shown by way of example, the sensor unit 43 is arranged in the same region along the cuvette 5 as the sensor unit 42. The bore 531 (not represented in FIG. 9) of the sensor connector 53 thus lies below the beam path 429 of the sensor unit 42. The sensor unit 43, or the sensor head 431 of the sensor unit 43, is for this purpose arranged in the recess 532 of the bottom 523 of the cuvette 5, or is inserted into the recess 523 by connecting the measuring unit 4 to the cuvette 5.

The sensor pins 411 of the sensor unit 41 protrude through the feed-throughs 511 of the socket 512, or of the cuvette 5, into the interior 57 of the cuvette. The sensor pins arranged in this way are used, for example, to determine the respiratory gas flow rate.

FIG. 10 shows a cross section along the cuvette 5, the section extending centrally through the bottom 523 and the cover 524. The Y-piece 54 is connected to the cuvette 5 by means of the coupling 60 of the cuvette 5. In order to allow leak-free connection between the Y-piece 54 and the cuvette 5, a seal 55 is arranged in the coupling 60. To prevent accidental release of the Y-piece 54 from the coupling 60, a retaining ring 58 is arranged in the coupling 60, and by way of example also serves as an axial bearing 56. For example, the retaining ring 58 and the Y-piece 54 are configured so that the Y-piece 54 is hooked behind the retaining ring 58 when being connected to the cuvette 5, and is therefore secured against accidental release.

Advantageously, the cuvette 5 in particular has a compact design so that the dead space volume in the cuvette 5 may be reduced, even with an attached Y-piece 54. The maximum length 70 of the cuvette 5, inclusive of the Y-piece 54 and the connector 59, is in this case 150 mm, preferably at most 80 mm, measured from the outermost edge of the cuvette 54 to the outermost edge of the connector 59, as represented in FIG. 11. In particular, the length 70 lies in a range of from 30 to 80 mm, although in some embodiments may also be less than 30 mm. In some embodiments, the length 70 is 46 (+/−15) mm. In some embodiments, for example, the Y-piece 54 has smaller dimensions so that the length 70 may be reduced. The connector 59 is, for example, configured as a standard connector for a patient interface 3 or a connection (for example a hose piece) to the patient interface 3. Depending on the embodiment of the patient interface 3, it is also possible to make the connector 59 correspondingly more compact or larger. The proportions of the connector 59 are therefore closely related to the patient interface 3 to be attached.

The outer diameter 73 of the connector 59 is also dependent on the selection of the patient interface 3. For example, the embodiment shown in FIG. 12a) is a standard connector for a tracheal cannula as the patient interface 3. Correspondingly, the outer diameter 73 of the connector 59 is 18.1 mm, this being suitable for example for a 15 mm inner cone. Depending on the patient interface 3, the outer diameter 73 may also be smaller or larger, however. If for example a simple small tube, through which the living being exhales into the cuvette 5, is used as the patient interface 3, the outer diameter 73 may be configured to be smaller in accordance with the small tube. In some embodiments, in particular the inner diameter of the connector 59 has a value at least similar to the inner diameter of the cuvette 5 in the region of the sensor connectors. If the inner cross section of the cuvette 5 and/or of the connector 59 is not circular, the cross-sectional areas should lie in a similar range, for example differing from one another by no more than 200 mm 2 and/or 50%.

The same requirements apply for the dimensions of the coupling 60, and these should in particular be configured so that, for example, a Y-piece 54 can be connected. In order to ensure a design of the cuvette 5 which is as compact as possible, the outer diameter 72 of the coupling is for example between 5 and 20 mm, these dimensions in this case being designed primarily for an intended use on living beings with small lung volumes (for example preterms). For use on living beings with larger lung volumes (and correspondingly also larger respiratory flow rates), larger diameters 72 (>20 mm) may also be necessary.

The inner faces 571a and 571b of the side walls 522a and 522b run parallel to one another with an exemplary spacing 71 of 4.2 mm. In general, the spacing 71 may be from 2 mm to 100 mm, preferably between 2 mm and 10 mm. Here again, the spacing 71 may be dimensioned to be larger depending on the living being. In some embodiments of the cuvette 5, the side walls 522a, 522b, or the inner faces 571a, 571b, are configured to be round instead of flat, so that the cuvette 5 has a circular cross section at least in the region of the sensor connectors 51, 52, 53, in which case the diameter would for example need to be dimensioned with the spacing 71.

A further view of the cuvette 5, inter alia with the lengths 74, 75, may be seen in FIG. 12b). The overall length 75 of the cuvette 5, inclusive of the coupling 60 and connector 59, is for example at most 120 mm, preferably less than 80 mm. In some embodiments, the overall length 75 of the cuvette 5 lies in a range of from 15 mm to 50 mm. In some exemplary embodiments, the length 75 of the cuvette 5 is preferably 36.5 (+/−15) mm.

The length 74 of the region of the sensor connectors 51, 52, 53 of the cuvette 5 is for example 5 (+/−0.4) mm, although it may also vary in a range of from 2 mm to 25 mm. In some embodiments of the measuring device 1, the length 74 also corresponds to the width 76 of the measuring unit 4, at least in the region of the housing sides 452, 454. The width 76 of the housing sides 452, 454 may in some embodiments also deviate by +/−10 mm from the length 74, and is for example 12 (+/−5) mm. In particular, the sensor connectors 51 and 53 are arranged next to one another in the bottom 523 of the cuvette 5 over the length 74. The length 74 is therefore also jointly crucially determined, but not limited, by the proportions of the sensor connectors 51, 53. The length 74 may therefore also be selected to be greater than would be necessary for the sensor connectors 51, 53. Overall, the length 74 should be selected at least so that it is sufficient to attach the measuring unit 42.

Since the Y-pieces generally comprise an extra connector for the pressure measurement but this measurement is ideally likewise integrated into the measuring device 1, in some embodiments it is possible to use a Y-piece which does not comprise this extra connector, so that further shortening of the construction is made possible.

It is to be understood that the dimensions indicated in respect of FIGS. 11 and 12a, b serve primarily for exemplary embodiments for living beings with small lung and/or tidal volumes (small animals, preterms, neonates, infants). In embodiments which for living beings with larger lung and/or tidal volumes (for example adult humans), it may sometimes be necessary to adapt the dimensions accordingly.

FIGS. 13 to 16 show further embodiments of the measuring device 1, partly in combination with a patient interface 3 and/or a ventilator apparatus 2. If the measuring device 1 is connected to the ventilator apparatus 2 only in such a way as to convey gas, that is to say the ventilator apparatus 2 is not also used for processing, calculation, evaluation, etc. of the measurement signals of the measuring unit 4, any ventilator apparatus may be used in conjunction with the measuring device 1 so long as the ventilator apparatus has a respiratory gas source. In some embodiments, the system is designed so that the measuring device 1 generates the measurement signals and if appropriate processes them by means of an internal conditioning unit, and these (conditioned and/or processed) measurement signals or measurement values are forwarded to the ventilator apparatus 2. The ventilator apparatus 2 is then, for example, configured and adapted to evaluate and/or output the measurement signals. For this purpose, the ventilator apparatus 2 has for example an interface for connection to the measuring device 1, or to the measuring unit 4, and the required evaluation and calculation units and if appropriate output units and/or display elements. Furthermore, it is also conceivable for the system to be configured so that the ventilator apparatus 2 automatically drives the respiratory gas source with the aid of the values and data of the measuring device 1 by means of a control unit and adapts the ventilation settings.

In some embodiments, the measuring device 1 is either configured so that it can be connected by means of the connector 59 either directly to a patient interface 3 and/or, as schematically represented in FIG. 13, is connected by means of a connection 6 to the patient interface 3. The connector 59 may, for example, also be adapted so that the measuring device 1, or the cuvette 5, is integrated directly into the patient interface 3 as a part thereof. In some embodiments, the patient interface 3 is also a part of the cuvette 5, for example if the patient interface 3 consists only of a mouthpiece through which the living being at least exhales. In order to evaluate and/or process the measurement signals of the measuring unit 4, or of the individual sensor units 41, 42, 43, the measuring unit 4 is for example equipped with at least one interface 44 by means of which a signal processing apparatus 10 is for example attached, the latter being configured by way of example to convert the measurement signals of the sensor units 41, 42, 43 at least into measurement values. In some embodiments, such a signal processing unit 10 is also already integrated into the measuring unit 4, as represented by way of example in FIG. 14. For example, the interface 44 is then used to forward the measurement values, for example to a display and/or to a ventilator apparatus 2, which is for example configured to evaluate and interpret the measurement values. As already described above, the measuring device 1, particularly the measuring unit 4, may itself also have conditioning, calculation and evaluation units and may if appropriate also comprise user interaction and display elements.

FIG. 15 shows an exemplary embodiment of the measuring device 1 in combination with a ventilator apparatus 2 and a patient interface 3. The cuvette 5 is by way of example connected by means of the coupling 60 to a Y-piece 54. The Y-piece 54 is in turn connected by means of the gas-conveying connections 7 and 9 to the ventilator apparatus 2. For example, the connection 7 is used as an inspiration line, that is to say respiratory gas from the ventilator apparatus 2 is conveyed through the Y-piece 54 and the cuvette 5 by means of the connector 59 and the connection 6 during the inspiration of the living being to the patient interface 3, to which for example a living being is attached. The connection 9 represents for example the expiration hose, via which the respiratory gas is conveyed away during the exhalation of the living being. During exhalation, the exhaled respiratory gas of the living being is thus conveyed from the patient interface 3 via the connection 6 to the connector 59, through the cuvette 5 and the Y-piece 54 and the connection 9, to the ventilator apparatus 2. For example, at least the respiratory gas flow rate, respiratory gas pressure and CO2 concentration of the respiratory gas are measured by the sensor units 41, 42, 43 both during the inhalation and during the exhalation. The measuring unit 4 is for example connected by means of an electrical connection 8 to the ventilator apparatus 2, which may at least evaluate and if appropriate interpret and output the measurement signals of the sensor units.

In some embodiments, the measuring device 1 is configured for example to be connected by means of an expiratory device 11 to a ventilator apparatus 2, as represented by way of example in FIG. 16. For this purpose, the expiratory device 11 is for example connected by means of the coupling 60 to the cuvette 5 in such a way as to convey gas. In contrast to the embodiment described in FIG. 15, the measuring device 1 is connected to the ventilator apparatus 2 only by means of a gas-conveying connection 7. For example, the living being is supplied with respiratory gas during inspiration by means of this gas-conveying connection 7. During the expiration, the exhaled respiratory gas of the living being is then for example released by means of the expiratory device 11, for example via a valve, to the ambient air.

In further supplementary or alternative embodiments, both the measuring unit 4 and the cuvette 5 may be arranged in and/or on the ventilator apparatus 2. For example, the measuring unit 4 is integrated into the ventilator apparatus 2 and the cuvette 5 can be externally attached to the measuring unit 4. For example, the cuvette 5 would therefore be arranged on the ventilator apparatus 2 while the measuring unit 4 is arranged in the ventilator apparatus 2. In some embodiments, the measuring unit 4 is located in the ventilator apparatus 2 such that the cuvette 5 is likewise integrated into the ventilator apparatus 2, so that it can be connected to the measuring unit 4. The entire measuring device is then located in the ventilator apparatus 2. Furthermore, it is also possible for the measuring device 1, consisting of the measuring unit 4 and the cuvette 5, to be fitted externally on the ventilator apparatus. For example, the measuring device 1 is for this purpose attached directly to a gas-conveying connector of the ventilator apparatus 2 and optionally fixed on the ventilator apparatus 2 by means of a holding mechanism.

It is, for example, also possible for the essential electronics component parts for the measuring unit 4 to be integrated into the ventilator apparatus 2 and for the measuring unit 4 to consist exclusively of the sensor units 41, 42, 43 (as well as any cabling and housing). Further component parts, for example the power supply, then lie in the ventilator apparatus 2. The measuring unit 4 may then be attached in the ventilator apparatus 2. Here, for example, a click solution by which the measuring unit 4 is fixed releasably in the ventilator apparatus 2 may be installed.

Is thus inter alia possible that, for example, the measuring unit 4 is integrated in the ventilator apparatus 2 (either firmly installed or easily removable/replaceable) and/or the measuring unit 4 is fitted on the ventilator apparatus 2 and (releasably) fixed and/or the measuring unit 4 is not fitted directly in or on the ventilator apparatus 2 but is arranged in the vicinity of the living being (for example a preterm/a patient). The same applies for the cuvette 5—it may for example be integrated together with the measuring unit 4 in the ventilator apparatus 2 and/or attached in the ventilator apparatus 2 to the measuring unit 4 and/or attached (externally) to the ventilator apparatus 2 to the measuring unit 4, which may optionally be arranged in or on the ventilator apparatus 2, and/or arranged—if appropriate together with the measuring unit 4—in the vicinity of the living being.

### LIST OF REFERENCE NUMERALS

1 measuring device
2 ventilator apparatus
3 patient interface
4 measuring unit
5 cuvette
6 connection
7 connection
8 cable
9 connection
10 signal processing apparatus
11 expiratory device
41 sensor unit
42 sensor unit
43 sensor unit
44 interface
45 housing
51 sensor connector
52 sensor connector
53 sensor connector
54 Y-piece
55 seal
56 axial bearing
57 interior
58 retaining ring
59 connector
60 coupling
61 connecting element
70 length (cuvette+Y-piece)
71 spacing
72 outer diameter (coupling)
73 outer diameter (connector)
74 length (sensor connectors)
75 length (cuvette)
76 width (measuring unit)
411 sensor pin
421 mirror
422 beam source
423 lens
424a prism
424b prism
425 lens
426 detector
426a detector face
426b detector face
427 surface
428a surface
428b face
429 beam path
431 sensor head
451 connecting element
452 housing side
453 housing bottom
454 housing side
511 feed-through
512 socket
521a outer face
521b outer face
522a side wall
522b side wall
523 bottom
524 cover
531 bore
532 recess
571a side face
571b side face
A angle
B continuation
C continuation
D angle

1.-70. (canceled)

71. A measuring device for analyzing a respiratory gas flow, wherein the measuring device comprising at least one measuring unit and a cuvette releasably connected to the measuring unit and adapted and configured for a respiratory gas to flow through it, and wherein
the measuring unit comprises at least two sensor units, at least one sensor unit being configured to determine a respiratory gas flow and at least one sensor unit being configured to determine a CO2 concentration in a respiratory gas,
the cuvette comprises at least two sensor connectors for attaching the sensor units for determining at least one respiratory gas flow and at least one CO2 concentration of a respiratory gas.

72. The measuring device of claim 71, wherein the cuvette comprises at least three sensor connectors, the at least third sensor connector being a sensor connector for a sensor unit for determining a respiratory gas pressure and/or an at least third sensor unit being a sensor unit for determining the respiratory gas pressure.

73. The measuring device of claim 71, wherein the sensor unit for determining the CO2 concentration in the respiratory gas comprises at least one radiation source, at least one detector unit, at least one mirror, at least two lenses and at least one prism.

74. The measuring device of claim 73, wherein the sensor unit for determining the CO2 concentration in the respiratory gas comprises at least two prisms, at least one of the at least two lenses being arranged on each prism.

75. The measuring device of claim 73, wherein the at least one mirror is a concave mirror which is coated for high reflection of radiation in a wavelength range of from 3500 nm to 4600 nm.

76. The measuring device of claim 73, wherein the at least one detector unit comprises at least two detector faces, at least one of the two detector faces being configured for detecting wavelengths in a wavelength range from 3950 nm to 4550 nm, and at least one of the at least two detector faces being configured for detecting wavelengths in a wavelength range from 3600 nm to 4200 nm, and/or at least one of the at least two detector faces being configured as measuring detector and at least one of the at least two detector faces being configured as comparison detector, the at least two detector faces having the same size and the detector face configured for detecting wavelengths in a wavelength range from 3950 nm to 4550 nm being configured as a measuring detector.

77. The measuring device of claim 73, wherein the radiation source is an infrared radiation source.

78. The measuring device of claim 73, wherein the at least one prism is configured as an inverting prism.

79. The measuring device of claim 73, wherein the cuvette is arranged between one of the at least two lenses and the at least one prism.

80. The measuring device of claim 73, wherein a total transmission of optics consisting of mirror, radiation source, lenses and prisms of the sensor unit as well as the cuvette is more than 50%.

81. The measuring device of claim 73, wherein the prisms, the lenses and the cuvette are made of a plastic, the plastic exhibiting a transmission of infrared rays at a material thickness of 2 mm of more than 85%.

82. The measuring device of claim 73, wherein the plastic consists of more than 90% by weight of a polysulfone, a polyethersulfone, a polymethylmethacrylate, a polycarbonate, a polytetrafluoroethylene, a poly(arylenesulfone), a polyimide, a polyamide and/or a mixture of at least one of the listed polymers and optionally a further polymer.

83. The measuring device of claim 73, wherein a surface of one of the prisms together with a surface of a second prism forms a tapering gap, the cuvette having an outer cross-section which fits the tapering gap in the area of a sensor connector for attaching the sensor unit for detecting the concentration of CO2.

84. The measuring device of claim 71, wherein the cuvette comprises a coupling for a gas-conveying connection to a patient interface.

85. The measuring device of claim 71, wherein the cuvette and the measuring unit are releasably connected to one another by at least one connecting element of the measuring unit and at least one connecting element of the cuvette.

86. The measuring device of claim 71, wherein the at least two sensor units of the measuring unit are arranged together in a housing.

87. The measuring device of claim 71, wherein the sensor unit for determining the respiratory gas pressure determines the respiratory gas pressure via a difference from atmospheric pressure.

88. The measuring device of claim 72, wherein the at least third sensor unit comprises at least one sensor head.

89. The measuring device of claim 71, wherein the measuring device, consisting of the measuring unit and the cuvette, has an overall weight of less than 120 g.

90. A system for analyzing a respiratory gas flow, wherein the system comprises at least
a. a ventilator apparatus,
b. a patient interface,
and wherein the system furthermore comprises the measuring device of claim 71, the measuring device being connected to the patient interface and the ventilator apparatus in a gas-conveying way.
